(19) United States
(12) Patent Application Publication
(10) Pub. No.: US 2004/0132802 A1
(43) Pub. Date: Jul. 8, 2004

(54) Pharmaceutical formulations and methods for modified release of statin drugs

(76) Inventors: Jackie Butler, Athlone (IE); John Devane, Athlone (IE); Paul Stark, Athlone (IE)

Correspondence Address: CROWELL & MORING LLP, INTELLECTUAL PROPERTY GROUP, P.O. BOX 14300, WASHINGTON, DC, 20044-4300, US

(21) Appl. No.: 10/653,469

(22) Filed: Sep. 3, 2003

Related U.S. Application Data
(60) Provisional application No. 60/407,270, filed on Sep. 3, 2002.

(51) Int. Cl. A61K 31/401; A61K 31/225

(52) U.S. Cl. 514/423; 514/460; 514/547

(57) ABSTRACT

The present invention is directed to compositions and methods of their use in treating, preventing, and/or managing one or more cardiovascular diseases using at least one poorly water-soluble statin, such as, for example, simvastatin and/or lovastatin. One method of the invention involves delaying release of the poorly water-soluble statin for a time sufficient to avoid metabolism of the statin at or near the gastrointestinal tract wall by the cytochrome P450 3A metabolic system, and releasing said statin in the ileum, colon, or both, with subsequent uptake into the hepatic portal vein and distribution to hepatocytes, wherein HMG-CoA reductase activity may be inhibited with minimal adverse drug interactions.

The present Application claims priority to U.S. Provisional Application No. 60/407,270 filed Sep. 3, 2002, the contents of which are incorporated herein in their entirety. The present invention is directed to compositions containing at least one poorly water-soluble statin compound, such as, for example, simvastatin and lovastatin, and methods of their use in treating, preventing, and/or managing one or more cardiovascular diseases.

The majority of the statin compounds are poorly water-soluble and significantly lipophilic. This is particularly true of the lactone statin drugs, including simvastatin and lovastatin. The lipophilicity of these drug compounds creates formulation difficulties that need to be addressed to improve the pharmaceutical effectiveness of these compounds.

Simvastatin is a lipid-lowering agent that is derived synthetically from fermentation product of Aspergillus terreus. After oral ingestion, simvastatin, in its inactive lactone form, is hydrolyzed to the corresponding beta-hydroxy acid form. The beta-hydroxy acid form is an inhibitor of 3-hydroxy-3-methylglutaryl-coenzyme A (HMG-CoA) reductase. This enzyme catalyzes the conversion of HMG-CoA to mevalonate, which is an early and rate-limiting step in the biosynthesis of cholesterol.

Simvastatin is commercially available under the brand name ZOCOR® for oral administration as 5 mg, 10 mg, 20 mg, 40 mg, and 80 mg tablets. It is generally used to reduce the risk of i) total mortality by reducing coronary death, ii) non-fatal myocardial infarction, iii) undergoing myocardial revascularization procedures, and iv) stroke or transient ischemic attack in patients with coronary heart disease and hypercholesterolemia. Package Insert ZOCOR®, 2002. Simvastatin is indicated for reducing total cholesterol (Total-C) low density lipoprotein cholesterol (LDL-C), apolipoprotein B and triglycerides, and increasing high density lipoprotein-cholesterol levels, in patients with primary hypercholesterolemia (heterozygous familial and nonfamilial) and mixed dyslipidemia (Fredrickson Type IIa and IIb). Id. It is also used to treat patients with hypertriglyceridemia (Fredrickson Type IV hyperlipidemia) and patients with primary dysbetalipoproteinemia (Fredrickson Type III hyperlipidemia). Id. Simvastatin is also used as an adjunct to other lipid lowering treatments (e.g., low density lipoprotein (apheresis). Id.

The cytochrome P450 isoform 3A4 (CYP3A4) system, which is the system primarily responsible for the metabolism of simvastatin, is expressed and active both in the liver and in the gut (de Waziers et al, 1990; Kolars et al, 1992(a), (b); Watkins, 1992; Kolars et al, 1994). The localization of CYP3A4 in the gut is not uniform (McKinnon & McManus, 1995, 1996; Windmill et al, 1997; Lown et al, 1997; Zhang et al, 1999), with higher levels of this iso-enzyme in the jejunum slightly low levels in the duodenum, and significantly lower levels in the ileum, cecum, and colon. ZOCOR®, which is a conventional immediate release formulation, releases drug predominantly in the stomach and upper small intestine, thereby presenting drug to the region of the small intestine with the highest levels of CYP3A4.

CYP3A4 mediates the oxidation of the fused ring system of lovastatin and simvastatin, transforming them into more polar metabolites. These polar metabolites may be absorbed less efficiently than the non-transformed parent compounds. Moreover, even if the polar metabolites are absorbed, they are extracted less efficiently into the hepatocytes due to their decreased lipophilicity. Therefore, metabolism of lovastatin or simvastatin in the gut by CYP3A4 results in less drug getting to the desired site of action. Thus, CYP3A4 activity attenuates therapeutic efficacy of the drug on cholesterol metabolism. In addition, statin hepatic availability may be increased by inhibiting CYP3A4 in the gut.

A number of the CYP3A4 metabolites, such as 6′ hydroxy, 6′-hydroxy-methyl, and 6′-exomethylene derivatives of lovastatin and simvastatin, may be hydrolyzed to their beta-hydroxy-acid forms by esterases in the liver or the plasma to become HMG-CoA reductase inhibitors. The presence of these metabolites in the systemic circulation increases the potential for undesired side effects due to their activity on HMG-CoA reductase.

Common adverse effects associated with HMG-CoA reductase inhibitor activity include muscle necrosis, manifesting as myalgia, limb weakness, elevation of serum creatinine kinase and myoglobinuria (Rhabdomylosis) (Hunninghake, 1992). Although myopathy is a rare occurrence (Tobert, 1988), severe myopathy has been seen in patients treated with simvastatin (Berland et al, 1991). Myopathy develops because HMG-CoA reductase inhibitors reduce the formation of mevalonate, which, in addition to being a precursor of cholesterol, is a precursor of ubiquinone. Ubiquinone is an essential component in the electron transport chain in mitochondria (Goldstein & Brown, 1990). FIG. 1 illustrates the biosynthesis of cholesterol and ubiquinone (Coenzyme Q). Thus, statins such as simvastatin and lovastatin not only interfere with the biosynthesis of cholesterol, but also with other metabolic pathways that require mevalonate. Thus, in non-hepatic tissues, such statins may exert undesirable effects on important metabolic pathways.

There exists a need in the art for formulations of poorly water soluble statins that limit the systemic exposure of the body to the statin compounds, and maximize hepatic-specific absorption of the drugs, thus increasing the efficacy of statin treatments and reducing undesirable side effects.

### BRIEF DESCRIPTION OF THE DRAWINGS

FIG. 1 illustrates the biosynthesis of cholesterol and ubiquinone (coenzyme Q). The figure is reproduced from Folkers K et al. Proc. Natl. Acad. Sci., 1990, 87, 8931. HMG-CoA reductase inhibitors inhibit the conversion of HMG-CoA to mevalonate. Mevalonate is a key precursor for both cholesterol and ubiquinone (Coenzyme Q). Depleted levels of ubiquinone in muscle tissue, including cardiac tissue, is believed to be the key factor associated with the rare, but serious and sometimes fatal, adverse events associated with HMG-CoA reductase inhibitor activity.

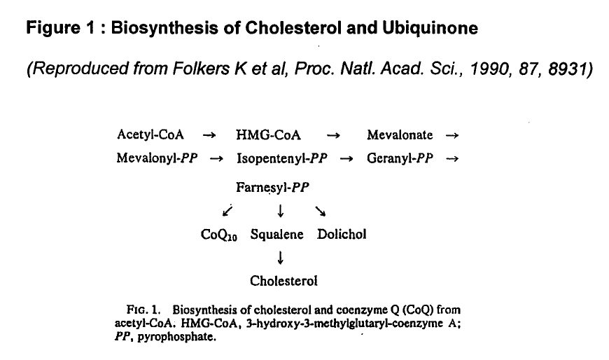

FIG. 2 illustrates how a conventional immediate release pharmaceutical formulation of simvastatin and/or lovastatin allows the penetration of polar metabolites into the systemic circulation. Penetration of polar metabolites of simvastatin and/or lovastatin into the systemic circulation and their subsequent conversion into active open acid forms results in the inhibition of ubiquinone biosynthesis in peripheral tissues. Depletion of ubiquinone levels in peripheral tissues is believed to be the main cause of the rare, but serious and sometimes fatal, adverse events of HMG-CoA reductase inhibitor activity.

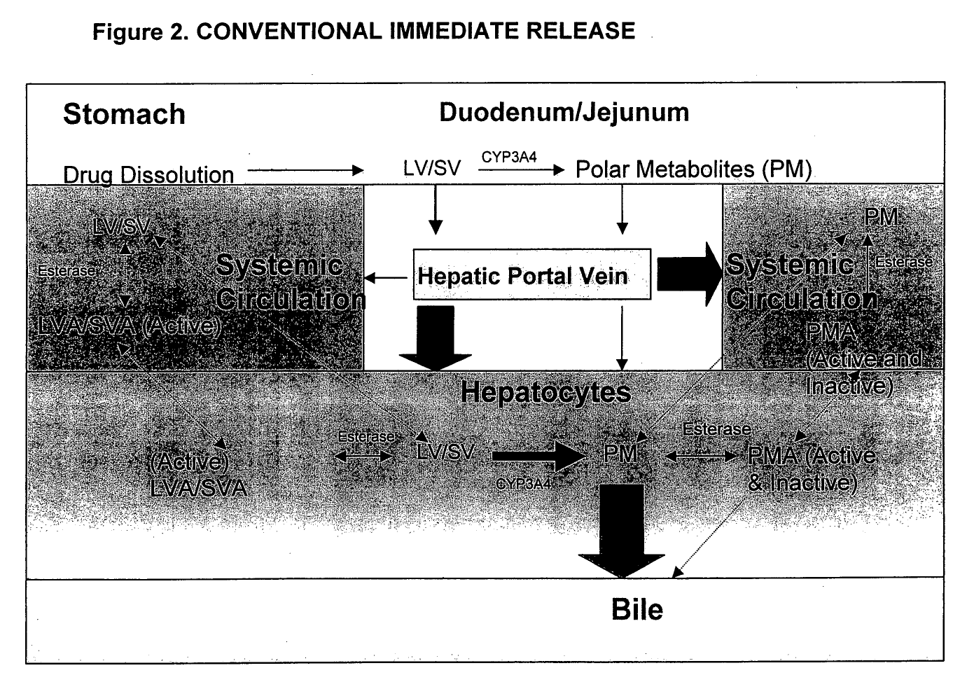

FIG. 3 illustrates how a modified release pharmaceutical formulation of simvastatin and/or lovastatin according to the present invention minimizes the penetration of polar metabolites into the systemic circulation.

### DETAILED DESCRIPTION OF THE PREFERRED EMBODIMENTS OF THE INVENTION

As used herein, the term “poorly water-soluble,” when used to refer to statin compounds including their lactone forms, their salt forms, or derivatives thereof, refers to a solubility of less than about 5 mg of compound in one liter of water. For example, poorly water-soluble statin compounds may have a solubility of less than about 1 mg of compound in one liter of water. Poorly water-soluble statin include, for example. simvastatin and lovastatin, derivatives thereof, and their pharmaceutically acceptable salts.

As used herein, the phrase “modified-release” formulation or dosage form includes a pharmaceutical preparation that achieves a desired release of the drug from the formulation. For example, a modified release formulation may extend the effect of a therapeutically effective dose of an active compound in a subject. Such formulations are referred to herein as “extended-release” formulations. In addition to maintaining therapeutic levels of the active compound, a modified release formulation may also be designed to delay the initiation of release of the active compound for a specified period. Such compounds are referred to herein as “delayed onset” formulations or dosage forms. Still further, modified-release formulations may exhibit properties of both delayed and extended release formulations, and thus be referred to as delayed-onset, extended-release formulations.

As used herein, the term “rapid” as used in the context of drug release, means that the formulation releases its drug component without any extension in the period of release. Generally, rapid release formulations will release their contents within from about 15 minutes to about 1 hour from administration.

In accordance with the present invention, rapid release formulations may be converted into modified release formulations by the addition of a functional coating. Such functional coatings generally delay the initiation of release of the drug. Once the delay has lapsed, the rapid release formulation is free to release it drug contents without any extension in release. Such formulations may be referred to as delayed-onset, rapid release.

“Extended” release, in the context of drug release, means that the formulation extends the period during which drug is released from the formulation. An extended release formulation may be prepared by including a polymer in the formulation to control the rate of dissolution or diffusion of the drug. Extended release formulations may further be coated with functional coatings to create a delay in the initiation of drug release. As noted above, such formulations may be referred to as delayed-onset, extended release.

As used herein, the term “pharmaceutically acceptable excipient” includes components that are compatible with the other ingredients in a pharmaceutical formulation and not injurious to the subject when administered in acceptable amounts.

As used herein, the term “pharmaceutically acceptable salt” includes salts that are physiologically tolerated by a patient. Such salts are typically prepare from an inorganic and/or organic acid. Examples of suitable inorganic acids include, but are not limited to, hydrochloric, hydrobromic, hydroiodic, nitric, sulfuric, and phosphoric acid. Organic acids may be aliphatic, aromatic, carboxylic, and/or sulfonic acids. Suitable organic acids include, but are not limited to, formic, acetic, propionic, succinic. Camphorsulfonic, citric, fumaric, gluconic, lactic, malic, mucic, tartaric, para-toluenesulfonic, glycolic, glucuronic, maleic, furoic, glutamic, benzoic, anthranilic, salicylic, phenylacetic, mandelic, pamoic, methanesulfonic, ethanesulfonic, pantothenic, benzenesulfonic (besylate), stearic, sulfanilic, alginic, galacturonic, and the like.

As used herein, the term “statin,” such as “simvastatin” and/or “lovastatin,” includes the particular statin compound, derivatives thereof, and any pharmaceutically acceptable salts thereof.

The phrase “therapeutically effective amount” of a statin, as used herein, refers to the amount of statin (or derivative or pharmaceutically acceptable salt thereof), which alone or in combination with other drugs, provides any therapeutic benefit in the prevention, treatment, and/or management of one or more cardiovascular diseases. Such diseases include, for example, coronary death, non fatal myocardial infarction, conditions resulting from myocardial revascularization procedures, stroke or transient ischemic attack in patients with coronary heart disease and/or hypercholesterolemia, elevated Total-C, LDL-C, Apo B, or TG, low HDL-C levels, primary hypercholesterolemia (for example, heterozygous familial or nonfamilial), mixed dyslipidemia (for example, Fredrickson Type IIa or IIb), hypertriglyceridemia (for example, Fredrickson Type IV hyperlipidemia), primary dysbetalipoproteinemia (for example Fredrickson Type III hyperlipidemia), and homozygous familial hypercholesterolemia. Other embodiments of the present invention comprise using the inventive formulations and methods of treatment as an adjunct to other lipid lowering treatments, for example, as an adjunct to LDL apheresis. The one or more diseases that can be treated, managed, and/or prevented by the formulations and/or methods of the present invention also include cardiovascular diseases that are not secondary to hypercholesterolemia.

In accordance with the present invention, simvastatin and/or lovastatin are provided in pharmaceutical formulations. The formulations of the present invention are designed, among other things, to increase the hepatic availability of these statins and/or minimize undesirable side effects typically associated with their use. For example, the formulations of the invention may minimize release of these statins in the stomach, duodenum, and/or jejunum, while optimizing uptake from the ileum and/or colon to the hepatic portal vein.

The present invention comprises a novel approach in the formulation and delivery of these statins from the GI tract to the liver, which optimizes the therapeutic effect and/or enhances safety of these drugs. For example, the formulations may be designed to minimize the intestinal CYP3A4 metabolism of the statins. This can enhance drug availability to the hepatocytes, and can improve the therapeutic efficacy of the formulations, while minimizing the peripheral exposure and the potential for unwanted side effects.

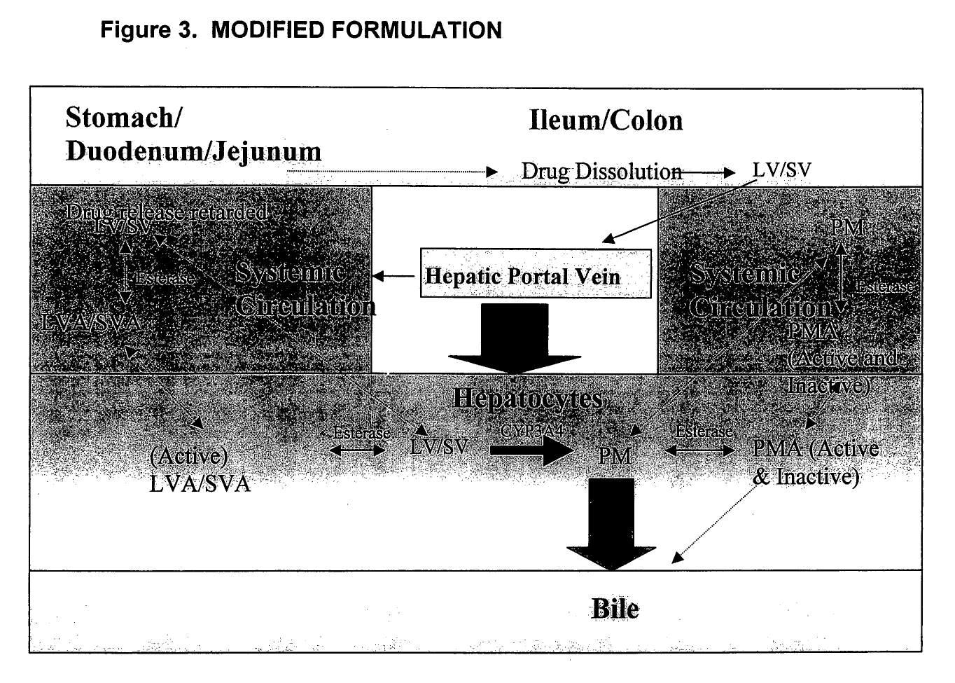

The formulations of the present invention may delay the release of the at least one statin until the formulation has passed out of the stomach, duodenum, and/or jejunum. and into the ileum and/or colon. FIG. 3 illustrates diagrammatically how the delayed release of the at least one statin may achieve more optimal absorption by the liver by having a slower input from the intestine to the hepatic portal vein, which results in a subsequent slower input from the hepatic portal vein to the hepatocytes. In this manner, the hepatic availability of the drug to the liver can be increased, while the systemic circulation of the drug is reduced. This provides a dose-sparing effect. In addition, the decrease in systemic circulation of these statins reduces undesirable side effects, such as those associated with undesirable depletion of ubiquinone in peripheral tissues.

As a result of this dose-sparing effect, the formulations of the present invention can utilize lower amounts of the statin component relative to conventional statin formulations. For example, the inventive formulations may include about 10 to about 100%, about 10 to about 85%, about 10 to about 70%, about 20 to about 70% about 20 to about 60%, or about 25 to about 50%, of the amount of statin contained in a conventional formulation of the drug. In one embodiment, the amount of simvastatin in the formulations of the present invention may be about 25% less than the amount of active ingredient in ZOCOR® needed to achieve a substantially similar therapeutic effect.

The present invention also provides methods and formulations for administering at least one statin at a low dose and in an extended-release formulation. In this way, clinically effective concentrations of the drug can be achieved in the liver itself, but clinically effective blood levels can be avoided in the peripheral or systemic circulation because 1) a significant portion of the drug is removed by the liver during first-pass, and/or 2) the relatively large volume of the systemic circulation compared with the smaller volume portal circulation creates a dilution effect.

The methods and formulations of the invention may involve the oral administration of at least one statin at a dose-delivery rate sufficient to provide a clinically effective blood level in the liver, but less than that required to provide a clinically effective blood level in the peripheral circulation. The dose delivery rate may be achieved by a modified release formulation.

Some embodiments of this invention involve administering a lactone HMG CoA reductase inhibitor in a manner that is selective for the liver, and that will reduce hypercholesterolemia without systemic depression of Coenzyme Q10 and its sequelae of muscle disease and other conditions, including those of the heart. These methods may involve use of an extended-release formulation of a low dose of a lactone HMG-CoA reductase inhibitor that is itself metabolized by the liver. Using methods and formulations of this invention, clinically effective blood levels of the HMG-CoA reductase inhibitor may be achieved in blood reaching the liver through the portal venous system, but undesirable levels in the peripheral blood circulation are avoided.

Liver-selective drug therapy, and its application to statin drugs, is discussed in WO 01/32162, which for its discussion of liver selective-therapy using station drugs is incorporated herein by reference.

The present invention can also provide advantages in that equivalent or higher, doses may be used, with better efficacy and/or fewer side effects observed. For example, simvastatin and lovastatin formulations of the present invention may include, for example, 100% to 200% of the amount of simvastatin and/or lovastatin in conventional formulations. However, even with these higher doses, formulations of the present invention achieve better efficacy and fewer side effects.

The delivery of the at least one statin to the ileum and colon provides optimal and enhanced/increased absorption of the drug compared to conventional immediate release compositions, wherein the drug may be released in the stomach, duodenum, and/or jejunum. Additionally, lovastatin and/or simvastatin may be administered to the intestine together with inhibitors of CYP3A4, which can advantageously be non-absorbable. Examples of suitable CYP3A4 inhibitors are disclosed in, for example. U.S. Pat. No. 6,028,054, to Benet et al, the relevant disclosure of which is incorporated herein by reference. Coadministration of these inhibitors with at least one statin further maximizes statin hepatic availability and further reduces the systemic side effects associated with ubiquinone depletion.

In addition, the methods and formulations of the present invention can be used to reduce drug interactions. For example, other drugs are known to inhibit CYP3A4. Such drugs include, but are not limited to, cyclosporine, ketoconazole, erythromycin, and HIV protease inhibitors, such as saquinavir. Thus, these drugs, if taken in combination with conventional simvastatin and lovastatin formulations, can produce undesirably high systemic levels of the statins. These high statin levels result in undesirable side effects associated with ubiquinone depletion. The present formulations and methods may reduce the possibility of these side effects.

In addition to the features of the present invention described above, the formulations may be designed to account for the hydrophobic nature of these statins. For example, the drug itself may be suitably processed, or it may be formulated with suitable excipients, to improve its solubility. As a result, the inventive formulations can provide a more efficient uptake of the drug in the ileum and/or colon.

As discussed above, the present inventive formulations can improve hepatic availability while reducing systemic effects. Thus, the therapeutic effect is improved, while unwanted side effects, such as those resulting from ubiquinone depletion, can be reduced.

The formulations of the present invention may be provided in a dosage form that is designed to modify the release of the drug. The modified-release may include a delay component, preventing release of the statin(s) until a desired location in the gastrointestinal tract is reached, or until a desired time has passed. The delay may range from about two to about eight hours, and this will be discussed in more detail below.

After the delay in release, a modified-release dosage form according to the present invention may release its contents immediately, or more gradually over an extended period. Thus, dosage forms may exhibit delayed-onset, rapid release, or delayed-onset, extended release properties.

Examples of suitable modified release formulations, which may be used in accordance with the present invention include, but are not limited to, matrix systems osmotic systems, ion exchange systems, membrane-controlled dosage forms, and soft gelatin capsules comprising solutions, microemulsions, emulsions, and/or precipitates. These formulations of the present invention may contain one or more statins and/or derivatives and/or pharmaceutically acceptable salts thereof. Suitable pharmaceutically acceptable salts are discussed above. Each of these types of dosage forms are briefly described below. A more detailed discussion of such forms may also be found in, for example The Handbook of Pharmaceutical Controlled Release Technology, D. L. Wise (ed.), Marcel Dekker, Inc., New York (2000); and also in Treatise on Controlled Drug Delivery: Fundamentals, Optimization, and Applications, A. Kydonieus (ed.), Marcel Dekker, Inc., New York, (1992).

Matrix-Based Dosage Forms

In some embodiments, the formulations of the present invention are provided as matrix-based dosage forms. To provide some overview, matrix formulations according to the invention may include hydrophilic, e.g., water-soluble, and/or hydrophobic, e.g., water-insoluble, polymers. The matrix formulations of the present invention may optionally be prepared with functional coatings, which may be enteric, e.g. exhibiting a pH-dependent solubility, or non-enteric, e.g., exhibiting a pH-independent solubility.

Matrix formulations of this invention may be prepared by using, for example, direct compression or wet granulation. A functional coating, as noted above, may then be applied in accordance with the invention. Additionally, a barrier or sealant coat may be applied over a matrix tablet core prior to application of a functional coating. The barrier or sealant coat may serve the purpose of separating an active ingredient from a functional coating, which may interact with the active ingredient, or it may prevent moisture from contacting the active ingredient. Detail of barriers and sealants are provided below. The description now turns to details of matrix formulations of this invention.

In a matrix-based dosage form, the simvastatin and/or lovastatin, and optional pharmaceutically acceptable excipients, are dispersed within a polymeric matrix, which typically comprises one or more water-soluble polymers and/or one or more water-insoluble polymers. The drug may be released from this dosage form by diffusion and/or erosion. Such matrix systems are described in detail by Wise and Kydonieus, supra.

Suitable water-soluble polymers include, but are not limited to, polyvinyl alcohol, polyvinylpyrrolidone, methylcellulose, hydroxypropylcellulose, hydroxypropylmethyl cellulose or polyethylene glycol, and/or mixtures thereof.

Suitable water-insoluble polymers include, but are not limited to, ethylcellulose, cellulose acetate cellulose propionate, cellulose acetate propionate, cellulose acetate butyrate, cellulose acetate phthalate, cellulose triacetate, poly (methyl methacrylate), poly (ethyl methacrylate), poly (butyl methacrylate), poly (isobutyl methacrylate), and poly (hexyl methacrylate), poly (isodecyl methacrylate), poly (lauryl methacrylate), poly (phenyl methacrylate), poly (methyl acrylate), poly (isopropyl acrylate), poly (isobutyl acrylate) poly (octadecyl acrylate), poly (ethylene), poly (ethylene) low density, poly (ethylene) high density, poly (ethylene oxide), poly (ethylene terephthalate), poly (vinyl isobutyl ether), poly (vinyl acetate), poly (vinyl chloride) and polyurethane, and/or mixtures thereof. Waxes, paraffins. and the like, are also included in this group.

Suitable pharmaceutically acceptable excipients include, but are not limited to, carriers, such as sodium citrate and dicalcium phosphate; fillers or extenders, such as stearates, silicas, gypsum, starches, lactose, sucrose, glucose, mannitol, talc, and silicic acid; binders, such as hydroxypropyl methylcellulose, hydroxymethyl-cellulose, alginates, gelatin, polyvinyl pyrrolidone, sucrose and acacia; humectants, such as glycerol; disintegrating agents, such as agar, calcium carbonate, potato and tapioca starch, alginic acid, certain silicates, EXPLOTAB™, crospovidone, and sodium carbonate; solution retarding agents, such as paraffin; absorption accelerators, such as quaternary ammonium compounds; wetting agents, such as sodium lauryl sulfate, cetyl alcohol, and glycerol monostearate; absorbents, such as kaolin and bentonite clay; lubricants, such as talc, calcium stearate, magnesium stearate, solid polyethylene glycols, and sodium lauryl sulfate; stabilizers, such as fumaric acid; coloring agents; buffering agents; dispersing agents; preservatives; organic acids; and organic bases. The aforementioned excipients are given as examples only and are not meant to include all possible choices. Additionally, many excipients may have more than one role, or be classified in mole than one group; the classifications are descriptive only, and not intended to limit any use of a particular excipient.

In one embodiment, a matrix-based dosage form comprises at least one statin; simvastatin and/or lovastatin; a filler, such as starch, lactose, or microcrystalline cellulose (AVICEL™); a binder/controlled-release polymer, such as hydroxypropy methylcellulose or polyvinyl pyrrolidone; a disintegrant, such as, EXPLOTAB™, crospovidone, or starch; a lubricant, such as magnesium stearate or stearic acid; a surfactant, such as sodium lauryl sulphate or polysorbate; and a glidant, such as colloidal silicon dioxide (AEROSIL™) or talc.

The amounts and types of polymers, and the ratio of water-soluble polymers to water-insoluble polymers in the formulations are generally selected to achieve a desired release profile of the at least one simvastatin and/or lovastatin, as described below. For example, by increasing the amount of water insoluble-polymer relative to the water soluble-polymer, the release of the drug may be delayed or slowed. This is due, in part, to an increased impermeability of the polymeric matrix, and, in some cases, to a decreased rate of erosion during transit through the GI tract.

Osmotic-Type Dosage Forms

In another embodiment, the modified release formulations of the present invention are provided as osmotic pump dosage forms. In an osmotic pump dosage form, a core containing the simvastatin and/or lovastatin and optionally one or more osmotic excipients is typically encased by a semipermeable membrane having at least one orifice. The semipermeable membrane is generally permeable to water, but impermeable to the drug. When the system is exposed to body fluids, water can generally penetrate through the semipermeable membrane into the core containing the drug and optional osmotic excipients. This causes the osmotic pressure to increase within the dosage form. Consequently, the drug is released through the orifice(s) in an attempt to equalize the osmotic pressure across the semipermeable membrane.

In more complex pumps, the dosage form may contain at least two internal compartments in the core. A first compartment contains the drug and the second compartment may contain a polymer which swells on contact with aqueous fluid. After ingestion, this polymer swells into the drug-containing compartment, diminishing the volume occupied by the drug, thereby delivering the drug from the device at a controlled rate over an extended period of time. Such dosage forms are often used when a zero order release profile is desired.

Osmotic pumps are well known in the art. For example, U.S. Pat. Nos. 4,088,864, 4,200,098, and 5,573,776, each of which is hereby incorporated by reference for this purpose, describe osmotic pumps and methods of their manufacture. The osmotic pumps useful in accordance with the present invention may be formed by compressing a tablet of an osmotically active drug, or an osmotically inactive drug in combination with an osmotically active agent, and then coating the tablet with a semipermeable membrane which is permeable to an exterior aqueous-based fluid but impermeable to the drug and/or osmotic agent.

One or more delivery orifices may be drilled through the semipermeable membrane wall. Alternatively, one or more orifices in the wall may be formed by incorporating at least one leachable pore-forming material in the wall. In operation, the exterior aqueous-based fluid is imbibed through the semipermeable membrane wall and contacts the drug to form a solution or suspension of the drug. The drug solution or suspension is then pumped out through the orifice as fresh fluid is imbibed through the semipermeable membrane.

Typical materials for the semipermeable membrane include semipermeable polymers known in the art to be useful in osmosis and reverse osmosis membranes, such as cellulose acylate, cellulose diacylate, cellulose triacylate, cellulose acetate, cellulose diacetate, cellulose triacetate, agar acetate, amylase triacetate, beta glucan acetate, acetaldehyde dimethyl acetate, cellulose acetate ethyl carbamate, polyamides, polyurethanes, sulfonated polystyrenes, cellulose acetate phthalate, cellulose acetate methyl carbamate, cellulose acetate succinate, cellulose acetate dimethyl aminoacetate, cellulose acetate ethyl carbamate, cellulose acetate chloracetate, cellulose dipalmitate, cellulose dioctanoate, cellulose dicaprylate, cellulose dipentanlate, cellulose acetate valerate, cellulose acetate succinate, cellulose propionate succinate, methyl cellulose, cellulose acetate p-toluene sulfonate, cellulose acetate butyrate, lightly cross-linked polystyrene derivatives, cross-linked poly(sodium styrene sulfonate), poly(vinylbenzyltrimethyl ammonium chloride), cellulose acetate, cellulose diacetate, cellulose triacetate, and/or mixtures thereof.

The osmotic agents that can be used in the pump are typically soluble in the fluid that enters the device following administration. Hydration of these agents produces an osmotic pressure gradient across the semipermeable wall. Suitable osmotic agents include, but are not limited to, magnesium sulfate, calcium sulfate, magnesium chloride, sodium chloride, lithium chloride, potassium sulfate, sodium carbonate, sodium sulfite, lithium sulfate, potassium chloride, sodium, sulfate, d-mannitol, urea, sorbitol, inositol, raffinose, sucrose, glucose. hydrophilic polymers such as cellulose polymers, and/or mixtures thereof.

As discussed above, the osmotic pump dosage form may contain a second compartment containing a swellable polymer. Suitable swellable polymers typically interact with water and/or aqueous biological fluids, which causes them to swell or expand to an equilibrium state. Acceptable polymers exhibit the ability to swell in water and/or aqueous biological fluids, retaining a significant portion of such imbibed fluids within their polymeric structure, so as to increase the hydrostatic pressure within the dosage form. The polymers may swell or expand to a very high degree, usually exhibiting a 2- to 50-fold volume increase. The polymers can be non-cross-linked or cross-linked. In one embodiment, the swellable polymers are hydrophilic polymers. Suitable polymers include, but are not limited to, poly(hydrox alkyl methacrylate) having a molecular weight of from 30,000 to 5,000.000; kappa-carrageenan; polyvinylpyrrolidone having a molecular weight of from 10,000 to 360,000; anionic and cationic hydrogels; polyelectrolyte complexes; poly(vinyl alcohol) having low amounts of acetate, cross-linked with glyoxal, formaldehyde, or glutaraldehyde. and having a degree of polymerization from 200 to 30,000; a mixture including methyl cellulose, cross-linked agar and carboxymethyl cellulose; a water-insoluble, water-swellable copolymer produced by forming a dispersion of finely divided maleic anhydride with styrene, ethylene, propylene, butylene or isobutylene; water-swellable polymers of N-vinyl lactams; and/or mixtures of any of the foregoing.

The term “orifice” as used herein comprises means and methods suitable for releasing the drug from the dosage form. The expression includes one or more apertures or orifices that have been bored through the semipermeable membrane by mechanical procedures. Alternatively, an orifice may be formed by incorporating an erodible element, such as a gelatin plug, in the semipermeable membrane. In such cases, the pores of the semipermeable membrane form a “passageway” for the passage of the drug. Such “passageway” formulations are described, for example, in U.S. Pat. No. Nos. 3,845,770 and 3,916,899.

The osmotic pumps useful in accordance with this invention may be manufactured by techniques known in the art. For example, the drug and other ingredients may be milled together and pressed into a solid having the desired dimensions (e.g., corresponding to the first compartment). The swellable polymer can then be formed, placed in contact with the drug, and both can be surrounded with the semipermeable agent. If desired, the drug component and polymer component may be pressed together before applying the semipermeable membrane. The semipermeable membrane may be applied by any suitable method, for example, by molding, spraying, or dipping.

Membrane-Controlled Dosage Forms

The modified release formulations of the present invention may also be provided as membrane-controlled formulations. Membrane-controlled formulations of the present invention can be made by preparing a rapid release core, which may be a monolithic (e.g., tablet) or multi-unit (e.g., pellet) type, and coating the core with a membrane. The membrane-controlled core can then be further coated with a functional coating. In between the membrane-controlled core and the functional coating, a barrier or sealant may be applied. With this as an overview, details of membrane-controlled dosage forms are provided below.

In one embodiment, the at least one simvastatin and/or lovastatin can be provided in a multiparticulate membrane controlled formulation. Simvastatin and/or lovastatin may be formed into an active core by applying the drug to a nonpareil seed having an average diameter in the range of about 0.4 to about 1.1 mm or about 0.85 to about 1.00 mm. The simvastatin and/or lovastatin may be applied with or without additional excipients onto the inert cores, and may be sprayed from solution or suspension using a fluidized bed coater (e.g., Wurster coating) or pan coating system. Alternatively, the simvastatin and/or lovastatin may be applied as a powder onto the inert cores using a binder to bind the simvastatin and lovastatin onto the cores. Active cores may also be formed by extrusion of the core with suitable plasticizers (described below) and any other processing aids as necessary.

The modified release formulations of the present invention may comprise at least one polymeric material, which can be applied as a membrane coating to the drug-containing cores. Suitable water-soluble polymers include, but are not limited to, polyvinyl alcohol, polyvinylpyrrolidone, methylcellulose, hydroxypropylcellulose, hydroxypropylmethyl cellulose or polyethylene glycol, and/or mixtures thereof.

Suitable water-insoluble polymers include, but are not limited to, ethylcellulose, cellulose acetate cellulose propionate, cellulose acetate propionate, cellulose acetate butyrate, cellulose acetate phthalate, cellulose triacetate, poly (methyl methacrylate), poly (ethyl methacrylate), poly (butyl methacrylate), poly (isobutyl methacrylate), and poly (hexyl methacrylate), poly (isodecyl methacrylate) poly (lauryl methacrylate), poly (phenyl methacrylate), poly (methyl acrylate), poly (isopropyl acrylate), poly (isobutyl acrylate), poly (octadecyl acrylate), poly (ethylene), poly (ethylene) low density, poly (ethylene) high density, poly (ethylene oxide), poly (ethylene terephthalate), poly (vinyl isobutyl ether), poly (vinyl acetate), poly (vinyl chloride) or polyurethane, and/or mixtures thereof.

EUDRAGIT™ polymers (available from Rohm Pharma) are polymeric lacquer substances based on acrylates and/or methacrylates. A suitable polymer that is freely permeable to the active ingredient and water is EUDRAGIT™ RL. A suitable polymer that is slightly permeable to the active ingredient and water is EUDRAGIT™ RS. Other suitable polymers which are slightly permeable to the active ingredient and water, and exhibit a pH-dependent permeability include, but are not limited to, EUDRAGIT™ L, EUDRAGIT™ S, and EUDRAGIT™ E.

EUDRAGIT™ RL and RS are acrylic resins comprising copolymers of acrylic and methacrylic acid esters with a low content of quaternary ammonium groups. The ammonium groups are present as salts and give rise to the permeability of the lacquer films. EUDRAGIT™ RL and RS are freely permeable (RL) and slightly permeable (RS), respectively, independent of pH. The polymers swell in water and digestive juices, in a pH-independent manner. In the swollen state, they are permeable to water and to dissolved active compounds.

EUDRAGIT™ L is an anionic polymer synthesized from methacrylic acid and methacrylic acid methyl ester. It is insoluble in acids and pure water. It becomes soluble in neutral to weakly alkaline conditions. The permeability of EUDRAGIT™ L is pH dependent. Above pH 5.0, the polymer becomes increasingly permeable.

In one embodiment comprising a membrane-controlled dosage form, the polymeric material comprises methacrylic acid co-polymers, ammonio methacrylate co-polymers, or a mixture thereof. Methacrylic acid co-polymers such as EUDRAGIT™ S and EUDRAGIT™ L (Rohm Pharma) are suitable for use in the controlled release formulations of the present invention, these polymers are gastroresistant and enterosoluble polymers. Their polymer films are insoluble in pure water and diluted acids. They dissolve at higher pHs, depending on their content of carboxylic acid. EUDRAGIT™ S and EUDRAGIT™ L can be used as single components in the polymer coating or in combination in any ratio. By using a combination of the polymers, the polymeric material may exhibit a solubility at a pH between the pHs at which EUDRAGIT™ L and EUDRAGIT™ S are separately soluble.

The membrane coating may comprise a polymeric material comprising a major proportion (i.e., greater than 50% of the total polymeric content) of one or more pharmaceutically acceptable water-soluble polymers, and optionally a minor proportion (i.e., less than 50% of the total polymeric content) of one or more pharmaceutically acceptable water insoluble polymers. Alternatively, the membrane coating may comprise a polymeric material comprising a major proportion (i.e., greater than 50% of the total polymeric content) of one or more pharmaceutically acceptable water insoluble polymers) and optionally a minor proportion (i.e., less than 50% of the total polymeric content) of one or more pharmaceutically acceptab water-soluble polymers.

Ammonia methacrylate co-polymers such as Eudragit RS and Eudragit RL (Rohm Pharma) are suitable for use in the controlled release formulations of the present invention. These polymers are insoluble in pure water, dilute acids, buffer solutions, or digestive fluids over the entire physiological pH range. The polymers swell in water and digestive fluids independently of pH. In the swollen state they are then permeable to water and dissolved actives. The permeability of the polymers depends on the ratio of ethylacrylate (EA), methyl methacrylate (MMA), and trimethylammoriioethyl methacrylate chloride (TAMCI) groups in the polymer. Those polymers having EA:MMA:TAMCI ratios of 1:2:0.2 (Eudragit RL) are more permeable than those with ratios of 1:2:0.1 (Eudragit RS). Polymers of Eudragit RL are insoluble polymers of high permeability, Polymers of Eudragit RS are insoluble films of low permeability.

The ammonio methacrylate co-polymers may be combined in any desired ratio. For example, a ratio of Eudragit RS:Eudragit RL (90:10) may be used. The ratios may furthermore be adjusted to provide a delay in release of the drug. For example, the ratio of Eudragit RS:Eudragit RL may be about 100:0 to about 80:20, about 100:0 to about 90:10. or any ratio in between. In such formulations, the less permeable polymer Eudragit RS would generally comprise the majority of the polymeric material.

The ammonio methacrylate co-polymers may be combined with the methacrylic acid co-polymers within the polymeric material in order to achieve the desired delay in release of the drug. Ratios of ammonio methacrylate co-polymer (e.g., Eudragit RS) to methacrylic acid co-polymer in the range of about 99:1 to about 20:80 may be used. The two types of polymers can also be combined into the same polymeric material, or provided as separate coats that are applied to a core.

In addition to the Eudragit polymers described above, a number of other such copolymers may be used to control drug release. These include methacrylate ester co-polymers (e.g., Eudragit NE 30D). Further information an the Eudragit polymers can be found in “Chemistry and Application Properties of Polymethacrylate Coating Systems,” in Aqueous Polymeric Coatings for Pharmaceutical Dosage Forms, ed, James McGinity, Marcel Dekker Inc., New York, pg 109-114).

The coating membrane may further comprise one or more soluble excipients so as to increase the permeability of the polymeric material. Suitably, the soluble excipient is selected from among a soluble polymer, a surfactant, an alkali metal salt, an organic acid, a sugar, and a sugar alcohol. Such soluble excipients include, but are not limited to, polyvinyl pyrrolidone, polyethylene glycol, sodium chloride, surfactants such as sodium lauryl sulfate and polysorbates, organic acids such as acetic acid, adipic acid, citric acid, fumaric acid, glutaric acid, malic acid, succinic acid, and tartaric acid, sugars such as dextrose, fructose, glucose, lactose and sucrose, sugar alcohols such as lactitol, maltitol, mannitol, sorbitol and xylitol, xanthan gum, dextrins, and maltodextrins. In some embodiments, polyvinyl pyrrolidone, mannitol, and/or polyethylene glycol can be used as soluble excipients. The soluble excipient(s) may be used in an amount of from about 1% to about 10% by weight, based on the total dry weight of the polymer.

In one embodiment, the polymeric material comprises one or more water-insoluble polymers, which are also insoluble in gastrointestinal fluids, and one or more water-soluble pore-forming compounds. For example, the water-insoluble polymer may comprise a terpolymer of polyviny polyvinylacetate, and/or polyvinylalcohol. Suitable water-soluble pore-forming compounds include, but are not limited to, saccharose, sodium chloride, potassium chloride, polyvinylpyrrolidone, and/or polyethyleneglycol. The pore-forming compounds may be uniformly or randomly distributed throughout the water insoluble polymer. Typically, the pore-forming compounds comprise about 1 part to about 35 parts for each about 1 to about 10 parts of the water insoluble polymers.

When such dosage forms come into contact with the dissolution media (e.g., intestinal fluids), the pore-forming compounds within the polymeric material dissolve to produce a porous structure through which the drug can diffuse. Such formulations are described in more detail in U.S. Pat. No. 4,557,925, which is herein incorporated by reference for this purpose. The porous membrane may also be coated with a functional coating, as described herein, to inhibit release in the stomach.

In one embodiment, such pore forming controlled release dosage forms comprise simvastatin and/or lovastatin; a filler, such as starch, lactose, or microcrystalline cellulose (AVICEL™); a binder/controlled release polymer, such as hydroxypropyl methylcellulose or polyvinyl pyrrolidone; a disintegrant, such as, EXPLOTAB™, crospovidone, or starch; a lubricant, such as magnesium stearate or stearic acid; a surfactant, such as sodium lauryl sulphate or polysorbates; and a glidant, such as colloidal silicon dioxide (AEROSIL™) or talc.

The polymeric material may also include one or more auxiliary agents such as fillers, plasticizers, and/or anti-foaming agents. Representative fillers include talc, fumed silica, glyceryl monostearate, magnesium stearate, calcium stearate, kaolin, colloidal silica, gypsum, micronized silica, and magnesium trisilicate. The quantity of filler used typically ranges from about 2% to about 300% by weight, and can range from about 20 to about 100%, based on the total dry weight of the polymer. In one embodiment, talc is the filler. In one embodiment, the anti-foaming agent is simethicone. The amount of anti-foaming agent used typically comprises from about 0% to about 0.5% of the final formulation.

The coating membranes, and functional coatings as well, can also include a material that improves the processing of the polymers. Such materials are generally referred to as plasticizers and include, for example, adipates, azelates, benzoates, citrates. isoebucates, phthalates, sebacates, stearates and glycols. Representative plasticizers include acetylated monoglycerides, butyl phthalyl butyl glycolate, dibutyl tartrate, diethyl phthalate, dimethyl phthalate, ethyl phthalyl ethyl glycolate, glycerin, ethylene glycol, propylene glycol, triacetin citrate, triacetin, tripropinoin, diacetin, dibutyl phthalate, acetyl monoglyceride, polyethylene glycols, castor oil, triethyl citrate, polyhydric alcohols, acetate esters, gylcerol triacetate, acetyl triethyl citrate, dibenzyl phthalate, dihexyl phthalate, butyl octyl phthalate, diisononyl phthalate, butyl octyl phthalate, dioctyl azelate, epoxidised tallate, triisoctyl trimellitate, diethylhexyl phthalate, di-n-octyl phthalate, di-1-octyl phthalate, di-1-decyl phthalate, di-n-undecyl phthalate, di-n-tridecyl phthalate, tri-2-ethylhexyl trimellitate, di-2-ethylhexyl adipate, di-2-ethylhexyl sebacate, di-2-ethylhexyl azelate, dibutyl sebacate, glyceryl monocaprylate, and glyceryl monocaprate. In one embodiment, the plasticizer is dibutyl sebacate. The amount of plasticizer used in the polymeric material typically ranges from about 10% to about 50%, for example, about 10, 20, 30, 40 or 50%, based on the weight of the dry polymer.

The amount of polymer to be used in the membrane-controlled formulations is typically adjusted to achieve the desired drug delivery properties, including the amount of drug to be delivered, the rate and location of drug delivery, the time delay of drug release, and the size of the multiparticulates in the formulation. The amount of polymer applied typically provides an about 10 to about 100% weight gain to the cores. In one embodiment, the weight gain from the polymeric material ranges from about 25 to about 70%.

The combination of all solid components of the polymeric material, including co-polymers, fillers, plasticizers, and optional excipients and processing aids, typically provides an about 10 to about 450% weight gain on the cores. In one embodiment, the weight gain is about 30 to about 160%.

The polymeric material can be applied by any known method, for example, by spraying using a fluidized bed coater (e.g., Wurster coating) or pan coating system. Coated cores are typically dried or cured after application of the polymeric material. Curing means that the multiparticulates are held at a controlled temperature for a time sufficient to provide stable release rates. Curing can be performed, for example, in an oven or in a fluid bed drier. Curing can be carried out at any temperature above room temperature.

A sealant or barrier can also be applied to the polymeric coatings, including both the functional coatings and the membrane coatings. A sealant or barrier layer may also be applied to the core prior to applying the polymeric membrane coating material. A sealant or barrier layer is not intended to modify the release of simvastatin and/or lovastatin. Suitable sealants or barriers are permeable or soluble agents such as hydroxypropyl methylcellulose, hydroxypropyl cellulose, hydroxypropyl ethylcellulose, and xanthan gum.

Other agents can be added to improve the processability of the sealar or barrier layer. Such agents include talc, colloidal silica, polyvinyl alcohol, titanium dioxide, micronized silica, fumed silica, glycerol monostearate, magnesium trisilicate and magnesium stearate, or a mixture thereof. The sealant or barrier layer can be applied from solution (e.g., aqueous) or suspension using any known means, such as a fluidized bed coater (e.g., Wurster coating) or pan coating system. Suitable sealants or barriers include, for example, OPADRY WHITE Y-1-7000 and OPADRY OY/B/28920 WHITE, each of which is available from Colorcon Limited, England.

The present invention also provides an oral dosage form comprising a multiparticulate simvastatin and/or lovastatin formulation as hereinabove defined, in the form of caplets, capsules, particles for suspension prior to dosing, sachets, or tablets. When the dosage form is in the form of tablets, the tablets may be disintegrating tablets, fast dissolving tablets, effervescent tablets, fast melt tablets, and/or mini-tablets. The dosage form can be of any shape suitable for oral administration of a drug, such as spheroidal, cube-shaped oval, or ellipsoidal. The dosage forms can be prepared from the multiparticulates in a manner known in the art and include additional pharmaceutically acceptable excipients, as desired.

Soft Gelatin Capsules

The formulations of the present invention may also be prepared as liquids, which may be filled into soft gelatin capsules. For example, the liquid may include a solution, suspension, emulsion, microemulsion, precipitate, or any other desired liquid media carrying the statin(s). The liquid may be designed to improve the solubility of the statin(s) upon release, or may be designed to form a drug-containing emulsion or dispersed phase upon release. Examples of such techniques are well known in the art. Soft gelatin capsules may be coated, as desired, with a functional coating to delay the release of the drug.

All of the embodiments described above, including but not limted to, the matrix-based, osmotic-based, membrane-controlled, and soft gelatin capsule forms, which may further take the form of monolithic and/or multi-unit dosage forms, may have a functional coating. Such coatings generally serve the purpose of delaying the release of the drug for a predetermined period. For example, such coatings may allow the dosage form to pass through the stomach and well down the intestine without being subjected to stomach acid or digestive juices. Thus, such coatings may dissolve or erode upon reaching a desired point in the gastrointestinal tract, such as the ileum or colon of the intestine.

Such functional coatings may exhibit pH-dependent or pH-independent solubility profiles. Those with pH-independent profiles generally erode or dissolve away a a predetermined period, and the period is generally directly proportional to the thickness of the coating. Those with pH-dependent profiles, on the other hand, may maintain their integrity while in the acid pH of the stomach, but quickly erode or dissolve upon entering the more basic upper intestine.

Thus, a matrix-based, osmotic-based, membrane-controlled, or soft gelatin capsule formulation may be further coated with a functional coating that delays the release of the drug. For example, a membrane-controlled formulation may be coated with a non-enteric coating that delays the exposure of the membrane-controlled formulation until the ileum or colon is reached. The non-enteric coating slowly dissolves as the dosage travels down the GI. When the coating is finally dissolved, the membrane-controlled formulation then is exposed to gastrointestinal fluid, and then releases the simvastatin and/or lovastatin over an extended period, in accordance with the invention. Examples of functional coatings such as these are well known to those in the art.

Thus, any of the modified-release formulations of the present invention may be designed to initially delay the release of the drug. Following the delay, the formulation may rapidly release the drug, or optionally, control the release for a specified period. For example, such formulations may minimize the release of the drug in the stomach, duodenum, and jejunum, while maximizing drug release in the ileum and colon. In general, this delay may be more than about 2 hours, for example about 2 to about 10 hours.

If desired, the at lease one statin provided in any of the formulations of the present invention may be processed to improve its solubility/dissolution. Such processing is particularly suitable for the poorly water soluble statins, such as simvastatin and lovastatin. In one embodiment, the statin drug is micronized. This is accomplished by conventional micronization techniques known to those of skill in the art, for example, jet milling, air jet milling, impact milling, media milling (aqueous or solvent), ball milling, pin milling, or fluid bed milling. In one embodiment of the invention, about 90% of the drug particles are less than about 20 microns in size. In another embodiment, about 50% of the drug particles are not more than about 10 microns in size. In some embodiments, particles of simvastatin and/or lovastatin are prepared as an even smaller, such as sub-micron, size.

Additionally, excipients may be included in the formulation to enhance the solubility/dissolution of the statin drugs. For example. surfactants, detergents, or any other agents that improve the dissolution of the statins may be included in the formulation. The formulations of this invention also contemplate incorporation of suitable excipients to maintain the integrity of particles of the active ingredient.

The present invention includes dosage forms having a modified dosage delivery and release. In some embodiments, formulations comprise a delayed onset combined with a rapid release formulation, wherein, for example, after an about 2 to about 8 hour delay more than 90% of the drug is released within about 15 minutes. Formulations of the present invention may also be designed to have an extended time of release, wherein, for example, after an about 2 to about 8 hour delay, more than 90% of the drug is released only after about 20 hours. In another embodiment of the present invention, a simvastatin formulation of the invention is designed to have the following modified release profile following delay of about 2 to about 8 hours: not more than about 50% of drug is released in about 1 hour, not more than about 75% of drug is released in about 2 hours, not less than about 20% of drug is released in about 4 hours, not less than about 30% of drug is released in about 6 hours, not less than about 40% of drug is released in about 8 hours, not less than about 50% of drug is released in about 10 hours, not less than about 60% of drug is released in about 12 hours, and not less than about 80% of drug is released in about 16 hours.

One of skill in the art is familiar with the techniques used to determine such dissolution profiles. The standard methodologies set forth in the U.S. Pharmacopeis, which is incorporated herein by reference in relevant part, may be used- For example, the dissolution profile may be measured in either a U.S. Pharmacopeia Type I Apparatus (baskets) or a U.S. Pharmacopeia Type II Apparatus (paddles). For pH-independent formulations, the formulations may be tested in phosphate buffer at pH 6.8, 37 C, and 50-100 rpm. For pH-dependent formulations, the formulations may be tested in 0.01-0.1 N HCl for the first 2 hours a 37 C and 50-100 rpm, followed by transfer to phosphate buffer at pH 6.8 or higher for the remainder of the test. Other buffer systems suitable for measuring the dissolution profile for pH-dependent and pH-independent formulations are well-known to those of skill in the art. To account for the hydrophobicity of the statins a surfactant, such as sodium lauryl sulfate, may be added in an amount sufficient to achieve sink conditions. Sufficient amounts can be determined empirically.

The methods of the invention may involve the oral administration of a pharmaceutical formulation at a dose-delivery rate sufficient to provide a clinically effective blood level in the liver, but less than that required to provide a clinically effective blood level in the peripheral circulation.

In general, the total daily dosage for treating hypercholesterolemic conditions described herein may range from about 0.1 mg to about 200 mg, about 0.1 mg to about 160 mg, about 0.1 mg to about 120 mg, or about 0.1 mg to about 100 mg, of at least one poorly water-soluble statin, such as, for example, simvastatin or lovastatin, or a pharmaceutically acceptable salt thereof. The daily dose may range from about 1 mg to about 80 mg, or from about 1 mg to about 40 mg. A single dose may be formulated to contain about 0.5, 1. 5, 10, 15, 20, 25, 30, 35, 40, 45, 50, 55, 60, 70, 80, 90, 100, 120, 150, or 200 mg of poorly water-soluble statin. The pharmaceutical compositions containing poorly water-soluble statin(s) may be administered in single or divided doses 1, 2, 3. 4, or more times each day. Alternatively, the dose may be delivered once every 2, 3, 4, 5, or more days. In one embodiment, the pharmaceutical compositions are administered once per day.

The therapeutic level is the minimum concentration of statin compound that is therapeutically effective in the patient. Of course, one of skill in the art will recognize that the therapeutic level may vary depending on the individual being treated and the severity of the condition. For example, the age, body weight, and medical history of the individual patient may affect the therapeutic efficacy of the therapy. A competent physician can consider these factors and adjust the dosing regimen to ensure the dose is achieving the desired therapeutic outcome without undue experimentation. It is also noted that the clinician and/or treating physician will know how and when to interpret, adjust, and/or terminate therapy in conjunction with individual patient response

Absolute systemic bioavailability from ZOCOR® is about 5%. Using the compositions of the present invention, systemic bioavailability of simvastatin and/or lovastatin may be reduced to below 5%, for example, about 4%, 3%, 2%, 1%, or 0%, or any amount less than 5%

Hepatic extraction of simvastatin from ZOCOR® is about 80%. Using the compositions of the present invention, hepatic extraction of simvastatin and/or lovastatin may be increased to greater than about 80%, for example, to about 85%, 90%, 95%, or 100%, or any amount above 80%.

Variability in AUC from ZOCOR® is about 50%. Using the compositions of the present invention, variability in AUC may be reduced to below about 50%, for example, to about 45%, 40%, 35%, 30%, 25%, or to about 20%, or any variability less than about 50%.

Any of the pharmaceutical compositions described above may further comprise one or more pharmaceutically active compounds other than a statin. Such compounds may be provided to treat the same condition being treated with the statin(s) of the present invention, or a different one. Those of skill in the art are familiar with examples of techniques for incorporating additional active ingredients into the formulations of the present invention. Alternatively, such additional pharmaceutical compounds may be provided in a separate formulation and co-administered to a patient with a statin-composition of the present invention. Such separate formulations may be administered before, after, or simultaneously with the administration of the statin(s).

The present invention is further illustrated by reference to the following examples. It will be apparent to those skilled in the art that many modifications, both to materials and methods, may be practiced without departing from the purpose and scope of the invention.

### EXAMPLES

### Example 1

### Production of Rapid Release Formulations of Simvastatin with Varying Amounts of Surface Active Agents, Prepared by Direct Compression

Rapid release formulations of simvastatin, comprising the components set forth in Table 1, are produced as follows:

TABLE 1

| | | Qty % | Qty % | Qty % |
|---|---|---|---|---|
| INGREDIENT | FUNCTION | (w/w) | (w/w) | (w/w) |
| SIMVASTATIN | Active | 5.00 | 5.00 | 5.00 |
| LACTOSE | Diluent | 50.28 | 48.78 | 45.78 |
| AVICEL PH101 | Dry Binder/ | 33.52 | 33.52 | 33.52 |
| | diluent | | | |
| SODIUM LAURYL | Surfactant | 0.5 | 2.0 | 5.0 |
| SULPHATE | | | | |
| SODIUM STARCH | Disintegrant | 10.00 | 10.00 | 10.00 |
| GLYCOLATE | | | | |
| (EXPLOTAB) | | | | |
| COLLOIDAL | Glidant | 0.20 | 0.20 | 0.20 |
| SILICON | | | | |
| DIOXIDE | | | | |
| MAGNESIUM | Lubricant | 0.50 | 0.50 | 0.50 |
| STEARATE | | | | |
| TOTAL | | 100 | 100 | 100 |

Each ingredient is weighed. The lactose, simvastatin, sodium lauryl sulphate, sodium starch glycolate, colloidal silicon dioxide, and avicel are mixed in a blender for 15 minutes. The magnesium stearate is added and the ingredients are mixed for a further 5 minutes. The mixture is then divided and compressed into tablets on a suitable tablet machine using plain oval tooling. The target weight of each tablet is 100 mg for 5.0 mg strength, 200 mg for 10.00 mg, or 400.00 mg for 20.00 mg.

### Example 2

### Production of Rapid Release Formulations of Simvastatin With Varying Amounts of Surface Active Agents, Prepared by Wet Granulation

Modified release formulations of simvastatin, comprising the components set forth in Table 2, are produced as follows:

TABLE 2

| | | Qty % | Qty % | Qty % |
|---|---|---|---|---|
| INGREDIENT | FUNCTION | (w/w) | (w/w) | (w/w) |
| SIMVASTATIN | Active | 5.00 | 5.00 | 5.00 |
| LACTOSE | Diluent | 45.28 | 48.78 | 40.78 |
| AVICEL PH101 | Dry Binder/ | 33.52 | 33.52 | 33.52 |
| | diluent | | | |
| SODIUM LAURYL | Surfactant | 0.5 | 2.0 | 5.0 |
| SULPHATE | | | | |
| SODIUM STARCH | Disintegrant | 10.00 | 10.00 | 10.00 |
| GLYCOLATE | | | | |
| (EXPLOTAB) | | | | |
| COLLOIDAL | Glidant | 0.20 | 0.20 | 0.20 |
| SILICON | | | | |
| DIOXIDE | | | | |
| MAGNESIUM | Lubricant | 0.50 | 0.50 | 0.50 |
| STEARATE | | | | |
| PVP | Binder | 5.00 | 5.00 | 5.00 |
| *ISOPROPYL | Solvent | N/A | N/A | N/A |
| ALCOHOL | | | | |
| TOTAL | | 100 | 100 | 100 |

*Removed during processing

Each of the ingredients is weighed. Simvastatin is dissolved in the isopropyl alcohol (IPA). The PVP is then dissolved in the IPA/simvastatin solution. In a suitable mixer (Planetary (Hobart), High Sher (Diosna/Fielder), 50% of the AVICEL, 50% of the lactose, and the sodium lauryl sulphate are blended for about 15 minutes to yield a homogeneous mixture. While the mixing is continued, the simvastatin/PVP solution is added. This is mixed until a suitable granulation end point is achieved. Additional IPA may be added, as needed, to produce a suitable granule. The granules are dryed (e.g., oven or fluidization equipment) until an acceptable level of moisture (<1,0%) and IPA (<0.5%) is achieved. The dry granules are passed through a suitably sized screen (100-500 micron) to select for the desired size granules and remove agglomerated granules.

### Example 3

### Production of Rapid Release Formulations of Simvastatin Containing Different Amounts of Disintegrant Direct Compression Method

The formulations set forth in Table 3 are produced according to the process of Example 1.

TABLE 3

| | | Qty % | Qty % | Qty % |
|---|---|---|---|---|
| INGREDIENT | FUNCTION | (w/w) | (w/w) | (w/w) |
| SIMVASTATIN | Active | 5.00 | 5.00 | 5.00 |
| LACTOSE | Diluent | 50.58 | 45.78 | 40.78 |
| AVICEL PH101 | Dry Binder/ | 33.72 | 33.52 | 33.52 |
| | diluent | | | |
| SODIUM STARCH | Disintegrant | 10.00 | 15.00 | 20.00 |
| GLYCOLATE | | | | |
| (EXPLOTAB) | | | | |
| COLLOIDAL | Glidant | 0.20 | 0.20 | 0.20 |
| SILICON | | | | |
| DIOXIDE | | | | |
| MAGNESIUM | Lubricant | 0.50 | 0.50 | 0.50 |
| STEARATE | | | | |
| TOTAL | | 100 | 100 | 100 |

### Example 4

### Production of Rapid Release Formulations of Simvastatin Containing Different Amounts of Disintegrant Direct Compression Method

The formulations set forth in Table 4 are produced according to the process of Example 2.

TABLE 4

| | | Qty % | Qty % | Qty % |
|---|---|---|---|---|
| INGREDIENT | FUNCTION | (w/w) | (w/w) | (w/w) |
| SIMVASTATIN | Active | 5.00 | 5.00 | 5.00 |
| LACTOSE | Diluent | 45.58 | 40.78 | 35.78 |
| AVICEL PH101 | Dry Binder/ | 33.72 | 33.52 | 33.52 |
| | diluent | | | |
| SODIUM STARCH | Disintegrant | 10.00 | 15.00 | 20.00 |
| GLYCOLATE | | | | |
| (EXPLOTAB) | | | | |
| COLLOIDAL | Glidant | 0.20 | 0.20 | 0.20 |
| SILICON | | | | |
| DIOXIDE | | | | |
| MAGNESIUM | Lubricant | 0.50 | 0.50 | 0.50 |
| STEARATE | | | | |
| PVP | Binder | 5.0 | 5.0 | 5.0 |
| *ISOPRYOYL | Solvent | N/A | N/A | N/A |
| ALCOHOL | | | | |
| TOTAL | | 100 | 100 | 100 |

*Removed during processing

### Example 5

### Production of Extended Release Matrix Formulations of Simvastatin Containing Methocel K100LV

The formulations set forth in Table 5 are produced according to the process of Example 1.

TABLE 5

| | | Qty % | Qty % | Qty % |
|---|---|---|---|---|
| INGREDIENT | FUNCTION | (w/w) | (w/w) | (w/w) |
| SIMVASTATIN | Active | 5.00 | 5.00 | 5.00 |
| LACTOSE | Diluent | 45.58 | 30.78 | 20.78 |
| AVICEL PH101 | Dry Binder/ | 28.72 | 23.52 | 13.52 |
| | diluent | | | |
| METHOCEL | Controlled | 20.00 | 40.00 | 60.00 |
| K110LV | Release | | | |
| PREMIUM CR | Polymer | | | |
| COLLOIDAL | Glidant | 0.20 | 0.20 | 0.20 |
| SILICON | | | | |
| DIOXIDE | | | | |
| MAGNESIUM | Lubricant | 0.50 | 0.50 | 0.50 |
| STEARATE | | | | |
| TOTAL | | 100 | 100 | 100 |

### Example 6

### Production of Extended Release Matrix Formulations of Simvastatin Containing Methocel K100LV and a Surface Active Agent

The formulations set forth in Table 6 are produced according to the process of Example 2.

TABLE 6

| | | Qty % | Qty % | Qty % |
|---|---|---|---|---|
| INGREDIENT | FUNCTION | (w/w) | (w/w) | (w/w) |
| SIMVASTATIN | Active | 5.00 | 5.00 | 5.00 |
| LACTOSE | Diluent | 45.58 | 30.78 | 20.78 |
| AVICEL PH101 | Dry Binder/ | 23.72 | 18.52 | 8.52 |
| | diluent | | | |
| METHOCEL | Controlled | 20.00 | 40.00 | 60.00 |
| K110LV | Release | | | |
| PREMIUM CR | Polymer | | | |
| COLLOIDAL | Glidant | 0.20 | 0.20 | 0.20 |
| SILICON | | | | |
| DIOXIDE | | | | |
| MAGNESIUM | Lubricant | 0.50 | 0.50 | 0.50 |
| STEARATE | | | | |
| PVP | Binder | 5.0 | 5.0 | 5.0 |
| *ISOPRYOYL | Solvent | N/A | N/A | N/A |
| ALCOHOL | | | | |
| TOTAL | | 100 | 100 | 100 |

*Removed during processing

### Example 7

### Production of Extended Release Matrix Formulations of Simvastatin Containing Methocel K100LV and a Surface Active Agent

The formulations set forth in Table 7 are produced according to the process of Example 1.

TABLE 7

| | | Qty % | Qty % | Qty % |
|---|---|---|---|---|
| INGREDIENT | FUNCTION | (w/w) | (w/w) | (w/w) |
| SIMVASTATIN | Active | 5.00 | 5.00 | 5.00 |
| LACTOSE | Diluent | 45.58 | 30.78 | 20.78 |
| AVICEL PH101 | Dry Binder/ | 27.72 | 22.52 | 12.52 |
| | diluent | | | |
| SODIUM LAURYL | Surfce Active | 1.00 | 1.00 | 1.00 |
| SULPHATE | Agent | | | |
| METHOCEL | Controlled | 20.00 | 40.00 | 60.00 |
| K110LV | Release | | | |
| PREMIUM CR | Polymer | | | |
| COLLOIDAL | Glidant | 0.20 | 0.20 | 0.20 |
| SILICON | | | | |
| DIOXIDE | | | | |
| MAGNESIUM | Lubricant | 0.50 | 0.50 | 0.50 |
| STEARATE | | | | |
| TOTAL | | 100 | 100 | 100 |

### Example 8

### Simvastatin Containing Methocel K100LV and a Surface Active Agent by Wet Granulation

The formulations set forth in Table 8 are produced according to the process of Example 2.

TABLE 8

| | | Qty % | Qty % | Qty % |
|---|---|---|---|---|
| INGREDIENT | FUNCTION | (w/w) | (w/w) | (w/w) |
| SIMVASTATIN | Active | 5.00 | 5.00 | 5.00 |
| LACTOSE | Diluent | 45.58 | 30.78 | 20.78 |
| AVICEL PH101 | Dry Binder/ | 22.72 | 17.52 | 7.52 |
| | diluent | | | |
| SODIUM LAURYL | Surfce Active | 1.00 | 1.00 | 1.00 |
| SULPHATE | Agent | | | |
| METHOCEL | Controlled | 20.00 | 40.00 | 60.00 |
| K110LV | Release | | | |
| PREMIUM CR | Polymer | | | |
| COLLOIDAL | Glidant | 0.20 | 0.20 | 0.20 |
| SILICON | | | | |
| DIOXIDE | | | | |
| MAGNESIUM | Lubricant | 0.50 | 0.50 | 0.50 |
| STEARATE | | | | |
| PVP | Binder | 5.0 | 5.0 | 5.0 |
| *ISOPROPYL | Solvent | N/A | N/A | N/A |
| ALCOHOL | | | | |
| TOTAL | | 100 | 100 | 100 |

*Removed during processing

### Example 9

### Production of Extended Release Matrix Formulations of Simvastatin Containing Methocel K100M

The formulations set forth in Table 9 are produced according to the process of Example 1.

TABLE 9

| | | Qty % | Qty % | Qty % |
|---|---|---|---|---|
| INGREDIENT | FUNCTION | (w/w) | (w/w) | (w/w) |
| SIMVASTATIN | Active | 5.00 | 5.00 | 5.00 |
| LACTOSE | Diluent | 45.58 | 30.78 | 20.78 |
| AVICEL PH101 | Dry Binder/ | 28.72 | 23.52 | 13.52 |
| | diluent | | | |
| METHOCEL | Controlled | 20.00 | 40.00 | 60.00 |
| K110LV | Release | | | |
| PREMIUM CR | Polymer | | | |
| COLLOIDAL | Glidant | 0.20 | 0.20 | 0.20 |
| SILICON DIOXIDE | | | | |
| MAGNESIUM | Lubricant | 0.50 | 0.50 | 0.50 |
| STEARATE | | | | |
| TOTAL | | 100 | 100 | 100 |

### Example 10

### Production of Extended Release Matrix Formulations of Simvastatin Containing Methocel K100M by Wet Granulation

The formulations set forth in Table 10 are produced according to the process of Example 2.

TABLE 10

| | | Qty % | Qty % | Qty % |
|---|---|---|---|---|
| INGREDIENT | FUNCTION | (w/w) | (w/w) | (w/w) |
| SIMVASTATIN | Active | 5.00 | 5.00 | 5.00 |
| LACTOSE | Diluent | 45.58 | 30.78 | 20.78 |
| AVICEL PH101 | Dry Binder/ | 23.72 | 18.52 | 8.52 |
| | diluent | | | |
| METHOCEL | Controlled | 20.00 | 40.00 | 60.00 |
| K110LV | Release | | | |
| PREMIUM CR | Polymer | | | |
| COLLOIDAL | Glidant | 0.20 | 0.20 | 0.20 |
| SILICON DIOXIDE | | | | |
| MAGNESIUM | Lubricant | 0.50 | 0.50 | 0.50 |
| STEARATE | | | | |
| PVP | Binder | 5.0 | 5.0 | 5.0 |
| *ISOPROPYL | Solvent | N/A | N/A | N/A |
| ALCOHOL | | | | |
| TOTAL | | 100 | 100 | 100 |

*Removed during processing

### Example 11

### Production of Extended Release Matrix Formulations of Simvastatin Containing Methocel K100M and a Surface Active Agent

The formulations set forth in Table 11 are produced according to the process of Example 1.

TABLE 11

| | | Qty % | Qty % | Qty % |
|---|---|---|---|---|
| INGREDIENT | FUNCTION | (w/w) | (w/w) | (w/w) |
| SIMVASTATIN | Active | 5.00 | 5.00 | 5.00 |
| LACTOSE | Diluent | 45.58 | 30.78 | 20.78 |
| AVICEL PH101 | Dry Binder/ | 27.72 | 22.52 | 12.52 |
| | diluent | | | |
| SODIUM LAURYL | Surfce Active | 1.00 | 1.00 | 1.00 |
| SULPHATE | Agent | | | |
| METHOCEL | Controlled | 20.00 | 40.00 | 60.00 |
| K110LV PREMIUM | Release | | | |
| CR | Polymer | | | |
| COLLOIDAL | Glidant | 0.20 | 0.20 | 0.20 |
| SILICON DIOXIDE | | | | |
| MAGNESIUM | Lubricant | 0.50 | 0.50 | 0.50 |
| STEARATE | | | | |
| TOTAL | | 100 | 100 | 100 |

### Example 12

### Production of Extended Release Matrix Formulations of Simvastatin Containing Methocel K100M and a Surface Active Agent by Wet Granulation

The formulations set forth in Table 12 are produced according to the process of Example 2.

TABLE 12

| | | Qty % | Qty % | Qty % |
|---|---|---|---|---|
| INGREDIENT | FUNCTION | (w/w) | (w/w) | (w/w) |
| SIMVASTATIN | Active | 5.00 | 5.00 | 5.00 |
| LACTOSE | Diluent | 45.58 | 30.78 | 20.78 |
| AVICEL PH101 | DryBinder | 22.72 | 17.52 | 7.52 |
| | diluent | | | |
| METHOCEL K100M | Controlled | 20.00 | 40.00 | 60.00 |
| PREMIUM CR | Release | | | |
| | Polymer | | | |
| SODIUM LAURYL | Surfactant | 1.00 | 1.00 | 1.00 |
| SULPHATE | | | | |
| COLLODIAL | Glidant | 0.20 | 0.20 | 0.20 |
| SILICON | | | | |
| DIOXIDE | | | | |
| MAGNESIUM | Lubricant | 0.50 | 0.50 | 0.50 |
| STEARATE | | | | |
| PVP | Binder | 5.0 | 5.0 | 5.0 |
| *ISOPROPYL | Solvent | N/A | N/A | N/A |
| ALCOHOL | | | | |
| TOTAL | | 100 | 100 | 100 |

### Example 13

### Addition of a pH-Dependent Delay Coating to Produce a Modified Release Simvastatin Formulation

Any of the formulations of the invention (e.g., those of Examples 1-12) may be further formulated to include a functional delay coating. The delay coating comprises a polymer, such as EUDRAGIT S™, which may be applied using suitable coating equipment (Accelacoata, Glatt, coating pan, etc.).

### Example 14

### pH-Independent Functional Coating Formulations

Any of the dosage forms according to the present invention (e.g., those of Examples 1-12) may be coated with a pH-independent coating, provided in the Table below.

| Ingredient | FUNCTION | g/Batch |
|---|---|---|
| EUDRAGIT RS 30D | Polymer | 200.00 |
| TALC | Antadherent | 60.00 |
| TRIETHYL CITRATE | Plasticizer | 12.00 |
| SIMETHICONE EMULSION | Dispersant | 1.00 |
| WATER | Solvent | 392.00 |
| TOTAL | | 665 |

### Example 15

### pH-Independent Functional Coating Formulations

Any of the dosage forms according to the present invention (e.g., those from Examples 1-12) may be coated with a pH-independent coating, provided in the Table below.

| Ingredient | FUNCTION | g/Batch |
|---|---|---|
| EUDRAGIT RS 12.5 | Polymer | 900 |
| EUDRAGIT RL 12.5 | Polymer | 300 |
| TALC | Antadherent | 105 |
| DIBUTYL SEBECATE | Plasticizer | 15 |
| MAGNESIUM STEARATE | Dispersant | 30 |
| ACETONE | Solvent | 825 |
| ISOPROPYL ALCOHOL | Solvent | 825 |
| TOTAL | | 3000 |

### Example 16

### Identifying Candidate Formulations for Clinical Trials

This Example provides a selection basis for screening possible candidates to proceed to clinical trials. This, of course, is not the only basis for choosing candidates, and other in vitro methods may be used to judge desirable formulations. Moreover, this screening step may be avoided altogether if desired, and all formulations may be tested directly in clinical trials.

Experiments are undertaken in four to eight dogs under general anesthesia induced with halothane and then maintained with ketamine and xylazine. Cardiovascular status is monitored by measurement of heart rate and blood pressure and by measurement of arterial blood gases. Ventilation is assisted to maintain blood gases within physiological limits. A catheter is placed in the femoral artery to permit sampling of arterial blood. After laparotomy, a catheter is placed in a mesenteric vein and advanced to the portal vein to permit sampling of portal venous blood samples.

Formulations of the invention are administered by mouth before the study begins, at a dose of 5 or 10 mg. Paired blood samples are then taken from systemic artery and portal vein at 0, 1, 2, 4, 6, 8, 10 and 12 hour points, measured from the time of administration of the formulation, for measurement of the blood concentration of the statin. The animals are sacrificed at the end of the experiment.

The systemic (as measured in femoral artery) concentration of the statin is compared to the liver concentration (as measured in portal vein), and candidate formulations are selected based upon a liver-selective effect.

### Example 17

### Assessing the Safety and Efficacy of Modified Release Formulations of Simvastatin

Following a four-week placebo period, where patients with primary hypercholesterolemia (Fredrickson Type 1 la and 1 lb) receive dietary advice, patients are randomized to receive:

A. A conventional simvastatin formulation, at 20 mg once daily for 6 weeks, and afterwards increasing to 40 mg daily for 6 weeks.

B. One of the formulations produced in Examples 13-15, at 5 mg daily for 6 weeks. At the end of that period, patients are randomized to receive either 5 mg or 10 mg of the formulation daily for a further 6 weeks.

C. The same formulation selected in (B), at 10 mg daily for 6 weeks. At the end of that period, patients are randomized to received either 10 mg or 20 mg daily for a further 6 weeks.

D. The same formulation selected in (B), at 20 mg for 6 weeks. At the end of that period, patients are randomized to receive either 20 mg or 40 mg daily for an additional 6 weeks.

Group A contains 20 patients, Groups B, C and D each contain 40 patients, permitting randomization into groups of 20 patients at week 6. This design permits a placebo, a dose-response comparison of the formulations of the present invention compared to conventional formulations.

Cholesterol levels are measured prior to study entry, prior to randomization (Baseline) and at weeks 1, 2, 4, 6, 8, 10 and 12. Systemic ubiquinone levels are measured prior to randomization and at weeks 6 and 12 to determine the relative depletion of systemic ubiquinone levels of the inventive formulation as compared to the conventional formulation. Plasma samples are also obtained for simvastatin analysis at weeks 6 and 12.

Efficacy endpoints which are measured include the change from baseline in total cholesterol (C), LDL-C, Triglycerides (TG), HDL-C, VLDL-C and the Total-C/HDL-C and LDL-C/HDL-C ratios. The change from baseline in systemic ubiquinone levels is also measured.

### Example 18

### Assessing the Safetv and Efficacy of Higher-Dose Modified Release Formulations of Simvastatin

Following a four-week placebo period, where patients with primary hypercholesterolemia (Frederickson Type IIa and IIb) receive dietary advice, patients are randomized to receive:

A. A conventional simvastatin formulation, at 20 mg once daily for 6 weeks and then increased to 40 mg once daily for 6 weeks and then increased to 80 mg once daily for 6 weeks; and

B. A modified release simvastatin formulation of the invention, at 20 mg once daily for 6 weeks and then increased to 40 mg once daily for 6 weeks and then increased to 80 mg once daily for 6 weeks.

Groups A and B contain 20 patients. This design permits a dose-response comparison of the formulations of the present invention compared to conventional formulations.

Cholesterol levels are measured prior to study entry, prior to randomization (Baseline) and at weeks 1, 2, 4, 6, 8, 10, 12, 14, 16 and 18. Systemic ubiquinone levels are measured prior to randomization and at week 6, 12, and 18, to determine the relative depletion of systemic ubiquinone levels of the inventive formulation as compared to the conventional formulation. Plasma samples are also obtained for simvastatin analysis at week 6, 12, and 18.

Efficacy endpoints which are measured include the change from baseline in total cholesterol®, LDL-C, Triglycerides (TG), HDL-C, VLDL-C and the Total-C/HDL-C and LDL-C/HDL-C ratios. The change from baseline in systemic ubiquinone levels is also measured.

### CITED DOCUMENTS

Berland Y, Coponat HV, Durand C, Baz M, Laugier R and Musso J L (1991) Rhabdomylosis with simvastatin use. Nephron 57, 365-366.

Cheng H, Rogers J D, Sweany A E, Dobrinska M R, Stei E A, Tate A C, Amin R D and Quan H (1992) Influence of age and gender on the plasma profiles of 3-hydroxy-3-methylglutaryl-coenzyme A (HMG-CoA) reductase inhibitory activity following multiple doses of lovastatin and simvastatin. Pharm Res 9 (12), 1629-1633.

Cheng H, Schwartz M S, Vickers S, Gilbert J D, Amin Rd, Depuy B, Liu L, Rogers J D, Pond S M, Duncan C A et al. (1994) Metabolic disposition pf simvastatin in patients with T-tube drainage. Drug Metab Dispos 22 (1), 139-142.

Desager J-P & Horsmans Y (1996) Clinical pharmacokinetics of 3-hydroxy-3-methylglutaryl-Coenzyme A reductase inhibitors. Clin Pharmacokinet 31 (5), 348-371.

deWaiziers I, Cugnenc P H, Yang C S, Leroux J P and Beaune P H (1990) Cytochrome P450 isoenzymes, epoxide hydrolase and gluthathione transferases in rat and human hepatic and extrahepatic tissues. J. Pharmacol. Expt. Ther. 253, 387-394.

Duggan D E & Vickers S (1990) Physiological disposition of HMG-CoA reductase inhibitors. Drug Metab Rev 22 (4), 333-362.

Folkers K, Langsjoen P, Willis R, Richardson P, Xia L-J, Ye C-Q and Tamagawa H (1990) Proc. Natl. Acad. Sci. USA 87, 8931-8934.

Goldstein J L & Brown M S (1990) Regulation of the mevalonate pathway. Nature 343, 425-430.

Halpin R A, Ulm E H, Till A E, Kari P H, Vyas K P, Hunninghake D B and Duggan D E (1993) Biotransformation of Lovastatin V: Species differences in in vivo metabolite profiles of mouse, rat, dog and human. Drug Metab Dispos 21 (6), 1003-1011.

Hamelin B A & Turgeon J (1998) Hydrophilicity/Lipophilicity: relevance for the pharmacology and clinical effects of HMG-CoA reductase inhibitors. TIPS 19, 26-37.

Hunninghake D B (1992) HMB CoA reductase inhibitors. Curr. Opin. Lipidol. 1, 22-28.

Kolars J C, Stetson P L, Rush B D, Ruwart M J, Schmiedlin-Ren P, Duell E A, Voorhees J J and Watkins P B (1992a) Cyclosporine metabolism by P450111A in rat enterocytes—another determinant of oral bioavailability? Transplantation 53(3), 596-602.

Kolars J C, Schmiedlin-Ren P. Schuetz J D, fang C and Watkins P B (1992b) Identification of rifampin-inducible P450111A4 (CYP3A4) in human small bowel enterocytes. J. Clin. Invest. 90 (5), 1871-1878.

Kolars J C, Lown K S, Schmiedlin-Ren P. Ghosh M, Fang C, Wrighton S A, Meriod R M and Watkins P B (1994) CYP3A gene expression in human gut epithelium. Pharmacogenetics 4 (5), 247-259.

Lown K S, Bailey D G, Fontana R J, Janardan S K, Adair C H, Fortlage L A, Brown M B, Guo W and Watkins P B (1997) Grapefruit juice increases felopipine oral availability in humans by decreasing CYP3A protein expression. J. Clin. Invest. 99 (10), 2545-2553.

Mauro V F (1993) Clinical pharmacokinetics and practical applications of simvastatin. Clin Pharmacokinet 24 (3), 195-202.

McKinnon & McManus (1995) Function and localization of cytochrome P450 involved in the metabolic activation of food-derived heterocyclic amines. Princess Takamatsu Symp 23, 145-153.

McKinnon & McManus (1996) Localization of cytochrome P450 in human tissues: implications for chemical toxicity. Pathology 28 (2), 148-155. Tobert JA (1988) Efficacy and long-term adverse effect pattern of lovastatin. Am. J. Cardiol. 62, 28]-34J.

Vickers S, Duncan C A, Vyas K P, Kari P H, Arison B, Prakash S R, Ramjit H G, Pitzenberger S M, Stokker G and Duggan D E (1990) In vitro and in vivo biotransformation of simvastatin, an inhibitor of HMB-CoA reductase. Drug Metab Dispos 18 (4), 476-483.

Wang R W, Kari P H, Lu A Y, Thomas P E, Guengerich F P and Vyas K P (1991) Biotransformation of lovastatin IV: Identification of cytochrome P450 3A proteins as the major enzymes responsible for the oxidative metabolism of lovastatin in rat and human liver microsomes. Arch Biochem Biophys 290 (2), 355-361.

Watkins P B (1992) Drug metabolism by cytochromes P450 in the liver and small bowel. Gastroenterol Clin North Am 21 (3), 511-526.

Windmill K F, McKinnon R A, Zhu X, Gaedigk A, Grant D M and McManus M E (1997) The role of xenophobic metabolizing enzymes in aryl amine toxicity and carcinogenesis: functional and localization studies. Mutate. Res. 376 (1-2), 153-160.

Zhang Q-Y, Dunbar D, Ostrowska A, Zeisloft S, Yang J and Kaminsky L S (1999) Characterization of human small intestinal Cytochrome P-450. Drug Metab Dispos 27 (7), 804-809.

What is claimed is:

1. A method for treating hypercholesterolemia comprising:
administering an oral pharmaceutical formulation comprising at least one poorly water-soluble statin;
delaying release of said statin for a time sufficient to avoid exposure of said statin to the stomach, duodenum, and jejunum; and
releasing said statin in the ileum, colon, or both.

2. The method according to claim 1, wherein said at least one poorly water-soluble statin is chosen from simvastatin, lovastatin, poorly water-soluble derivatives thereof, and pharmaceutically acceptable salts thereof.

3. The method according to claim 1, wherein said oral pharmaceutical formulation further comprises at least one formulation that enhances the delivery characteristics of the at least one poorly water-soluble statin.

4. The method according to claim 3, wherein said oral pharmaceutical formulation is prepared in a manner comprising at least one of a) reduction of particle size to sizes ranging from micrometer to nanometer sized particles, b) inclusion of the at least one poorly water-soluble statin in a solid/liquid dispersion, emulsion, or microemulsion, and c) incorporation of at least one surface-active agent in the formulation.

5. The method according to claim 1, wherein the release of the at least one poorly water-soluble statin is delayed for more than about 2 hours from administration,

6. The method according to claim 1, wherein the release of the at least one poorly water-soluble statin is delayed for more than about 4 hours from administration.

7. The method according to claim 5, wherein the release of the at least one poorly water-soluble statin occurs over a period of less than about 2 hours.

8. The method according to claim 5, wherein the release of the at least one poorly water-soluble statin occurs over a period of greater than about 2 hours.

9. The method according to claim 8, wherein the release of the at least one poorly water-soluble statin occurs over a period of greater than about 4 hours.

10. The method according to claim 1, wherein said oral pharmaceutical formulation further comprises at least one non-absorbable inhibitor of CYP3A4.

11. The method according to claim 10, wherein said at least one non-absorbable inhibitor of CYP3A4 and said at least one statin are both released rapidly.

12. The method according to claim 10, wherein said at least one non-absorbable inhibitor of CYP3A4 and said at least one statin are both released in an extended manner.

13. The method according to claim 1, wherein the oral pharmaceutical formation achieves a therapeutic effect with a daily dose comprising 80 mg or less of said at least one statin.

14. The method according to claim 13, wherein the oral pharmaceutical formulation achieves a therapeutic effect with a daily dose comprising 40 mg or less of said at least one stain.

15. The method of claim 1, wherein the formulation is administered to a subject and the subject's serum low density lipoprotein-cholesterol (LDL-C) levels are reduced following the administration.

16. The method of claim 1, wherein the formulation is administered to a subject and the subject's serum high density lipoprotein-cholesterol (HDL-C) levels are increased following the administration.

17. A method of treating one or more cardiovascular diseases comprising administering, to a subject in need of treatment, a therapeutically effective amount of at least one poorly water soluble statin chosen from simvastatin and lovastatin, or a pharmaceutically acceptable salt thereof, in a modified release formulation, wherein the subject obtains a therapeutic benefit resulting from the administration of the at least one statin, and wherein the amount of the at least one statin, or pharmaceutically acceptable salt thereof, is less than the amount required to achieve the same therapeutic benefit using a conventional immediate release formulation of the at least one statin.

18. A method of reducing one or more side effects associated with administration of at least one poorly water soluble statin chosen from simvastatin and lovastatin, comprising administering a therapeutically effective amount of the at least one statin or a pharmaceutically acceptable salt thereof, to a subject in need of such reduction in side effects, wherein one or more side effects are reduced relative to those resulting from the administration of an equivalent amount of a conventional immediate release formulation of the at least one statin.

19. The method according to claim 18, comprising administering a dose of the at least one statin that is equivalent or higher than a conventional immediate release dose formulation of the at least one statin.

20. The method according to claim 18, comprising administering a dose of the at least one statin of about 0.1 to about 200 mg.

21. The method according to claim 20. comprising administering a dose of the at least one statin of about 0.1 to about 120 mg.

22. The method according to claim 1, wherein the at least one statin is administered at a dose-delivery rate sufficient to provide a clinically effective blood level in the liver, but less than that required to provide a clinically effective blood level in the peripheral circulation.

23. The method of claim 1, wherein the oral pharmaceutical formulation exhibits a release rate of the at least one poorly water-soluble statin, as measured in a Type I dissolution apparatus, in a pH 6.8 buffer, of the following:
a delay in release of about 2 to about 8 hours
1 hours (after the delay): about 0 to about 50%
2 hours (after the delay): less than about 75%
4 hours (after the delay): greater than about 20%
6 hours (after the delay): greater than about 40%
8 hours (after the delay): greater than about 60%.

24. The method of claim 1, wherein the formulation is administered to a subject to treat one or more cardiovascular diseases or conditions that are secondary to the hypercholesterolemia.

25. A method for treating one or more cardiovascular diseases or conditions that are not secondary to hypercholesterolemia comprising:
administering an oral pharmaceutical formulation comprising at least one poorly water-soluble statin;
delaying release of said statin for a time sufficient to avoid exposure of said statin to the stomach, duodenum, and jejunum; and releasing said statin in the ileum, colon, or both.

26. The method according to claim 25 wherein said at least one poorly water-soluble statin is chosen from simvastatin, lovastatin, poorly water-soluble derivatives thereof, and pharmaceutically acceptable salts thereof.

27. The method according to claim 25, wherein the oral pharmaceutical formulation achieves a therapeutic effect with a daily dose ranging from about 0.1 to about 200 mg of said at least one statin.

28. The method of claim 25 wherein the oral pharmaceutical formulation comprises a polymeric coating.

29. The method of claim 28, wherein the polymeric coating is an enteric, erodible, diffusion-controlled or dissolution-controlled coating.

30. The method of claim 25, wherein the oral pharmaceutical formulation is administered to a subject, and the subject's serum high density lipoprotein cholesterol (HDL-C) levels are increased following the administration.

31. The method of claim 25, wherein the oral pharmaceutical formulation is administered to a subject, and the subject's serum low density lipoprotein cholesterol (LDL-C) levels are reduced following the administration.

32. A pharmaceutical formulation for oral administration comprising a therapeutically effective amount of at least one poorly water-soluble statin;
means for preventing the release of the at least one poorly water-soluble statin in the stomach, duodenum, and jejunum; and means for optimizing the uptake of the at least one poorly water-soluble statin in the ileum, colon, or both.

33. The formulation according to claim 32, wherein said at least one poorly water-soluble statin is chosen from simvastatin, lovastatin, poorly water-soluble derivatives thereof, and pharmaceutically acceptable salts thereof.

34. The formulation according to claim 32, wherein the formulation achieves a therapeutic effect with a daily dose ranging from about 0.1 to about 200 mg of said at least one statin.

35. The formulation of claim 32, wherein the formulation comprises a polymeric coating.

36. The formulation of claim 35, wherein the polymeric coating is an enteric, erodible, diffusion-controlled or dissolution-controlled coating.

37. The formulation of claim 32, wherein the formulation is administered to a subject, and the subject's serum high density lipoprotein-cholesterol (HDL-C) levels are increased following the administration.

38. The formulation of claim 32, wherein the formulation is administered to a subject, and the subject's serum low density lipoprotein-cholesterol (LDL-C) levels are reduced following the administration.

39. A formulation comprising:
a matrix comprising a therapeutically effective amount of at least one poorly water-soluble statin, at least one surface-active agent, and at least one water-soluble or water-permeable polymer.

40. The formulation according to claim 39, wherein said at least one poorly water-soluble statin is chosen from simvastatin, lovastatin. poorly water-soluble derivatives thereof, and pharmaceutically acceptable salts thereof.

41. The formulation according to claim 39. wherein the formulation achieves a therapeutic effect with a daily dose ranging from about 0.1 to about 200 mg of said at least one statin.

42. A formulation comprising:
a core comprising a therapeutically effective amount of at least one poorly water-soluble statin and at least one surface-active agent; and
a polymeric membrane-controlled coating comprising less than 50% by weight of at least one water-soluble or water-permeable polymer and greater than 50% by weight of at least one water-insoluble or water-impermeable polymer.

43. The formulation according to claim 42, wherein said at least one poorly water-soluble statin is chosen from simvastatin, lovastatin, poorly water-soluble derivatives thereof, and pharmaceutically acceptable salts thereof.

44. The formulation according to claim 42, wherein the formulation achieves a therapeutic effect with a daily dose ranging from about 0.1 to about 200 mg of said at least one statin

45. The method of claim 1, wherein the formulation inhibits HMG-CoA reductase activity in the liver,

46. The method of claim 25, wherein the formulation inhibits HMG-CoA reductase activity in the liver.
